(19) United States
(12) Patent Application Publication
(10) Pub. No.: US 2009/0082572 A1
(43) Pub. Date: Mar. 26, 2009

(54) PROCESS FOR AMORPHOUS ESOMEPRAZOLE

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad Andhrapradesh (IN)

(21) Appl. No.: 11/718,273

(22) PCT Filed: Nov. 14, 2005
(86) PCT No.: PCT/IN2005/000367

§ 371 Date: Apr. 30, 2007

(51) Int. Cl. C07D 401/12

(52) U.S. Cl. 546/273.7

(57) ABSTRACT

The present invention relates to a commercially viable process for preparation of amorphous esomeprazole. Thus, amorphous esomeprazole is prepared by suspending esomeprazole in water and then subjecting the suspension to lyophilization at −70° C.

### FIELD OF THE INVENTION

The present invention relates to a commercially viable process for preparation of amorphous esomeprazole.

### BACKGROUND OF THE INVENTION

Omeprazole, chemically 5-methoxy-2-[[(4-methoxy-3,5-dimethyl-2-pyridinyl)methyl]sulfinyl]-1H-benzimidazole and its therapeutic uses are disclosed in European Patent No. 5129. Omeprazole is a well-known gastric acid secretion inhibitor, and is useful as an anti ulcer agent. Omeprazole has a stereogenic center at sulfur and therefore exist as two optical isomers such as R-omeprazole and S-omeprazole (esomeprazole).

The salts of the enantiomers of omeprazole are described in WO 94/27988. PCT Publication No. WO 98/28294 disclosed esomeprazole in an amorphous form, a partly crystalline form A, and a substantially crystalline form B.

PCT Publication No. WO 2004/076440 A1 described crystalline forms, Form I and Form II, of esomeprazole, and its hydrates. PCT Publication No. WO 2004/020436 A1 described amorphous hydrates of esomeprazole magnesium and process for their preparation. PCT Publication No. WO 2004/002982 A2 described amorphous form of esomeprazole free base and process for its preparation.

U.S. Pat. No. 6,369,085 described crystalline forms of esomeprazole magnesium, esomeprazole magnesium dihydrate, esomeprazole magnesium trihydrate and esomeprazole potassium.

The alkaline salts of (S)-enantiomer of omeprazole (esomeprazole), the pharmaceutical preparations of these salts and the method of treatment of gastric acid-related diseases using them are disclosed in U.S. Pat. No. 4,738,974, U.S. Pat. No. 5,877,192 and U.S. Pat. No. 5,714,504.

PCT Application No. PCT/IN05/00197 describes an amorphous form of esomeprazole. Even though the process described in the patent application yields amorphous esomeprazole in Laboratory scale, we have found that there is a problem in the scale up of process. The main problem in the scale up of this process is that the development of color in the product during drying even under reduced pressure of wet product obtained.

The object of the present invention is to provide commercially viable process for pure amorphous esomeprazole.

### DETAILED DESCRIPTION OF THE INVENTION

In accordance with the present invention, there is provided a process for amorphous form of esomeprazole.

In accordance with the present invention, a process is provided for the preparation of amorphous esomeprazole, which comprises:
a) suspending esomeprazole in water; and
b) subjecting the suspension obtained in step-(a) to lyophilization to remove water.

Lyophilization is preferably carried out at about −20° C. to −80° C. and more preferably at about −40° C. to −70° C.

The esomeprazole used in the process may be in any polymorphic form, hydrated form etc., can be prepared by known techniques.

The invention will now be further described by the following examples, which are illustrative rather than limiting.

### EXAMPLE 1

Esomeprazole (50 gm) was suspended in water (100 ml) at 25° C. and then stirred at the same temperature for 3 hours. Subsequently, the water was removed by lyophilization at about −70° C. After isolation from the lyophilization vessel there were obtained 49.7 gm of amorphous esomeprazole (HPLC Purity: 99.89%, water content: 2.0%).

### EXAMPLE 2

Tetrahydrofuran (250 ml) and water (500 ml) were added to esomeprazole potassium salt (50 gm) at 25-30° C., cooled to 20° C. and then the pH is adjusted to 7.5-8.0 with acetic acid. The reaction mass was cooled to 5° C., stirred for 2 hours at 0-5° C., filtered the mass, washed with 50 ml of chilled mixture of water and tetrahydrofuran (2:1) and again washed with water (100 ml). To the wet cake obtained was added water (100 ml) at 25° C. and then stirred for 30 minutes. Subsequently, the water was removed by lyophilization at about −60° C. After isolation from the lyophilization vessel there were obtained 26.2 gm of amorphous esomeprazole (HPLC Purity: 99.87%, water content: 2.5%).

1. A process for preparation of amorphous esomeprazole, which comprises:
a) suspending esomeprazole in water; and
b) subjecting the suspension obtained in step-(a) to lyophilization.

2. The process as claimed in claim 1, wherein the lyophilization is carried out at about −20° C. to −80° C.

3. The process as claimed in claim 2, wherein the lyophilization is carried out at about −40° C. to −70° C.
